SEQUENCE LISTING (2 sequences)

SEQ ID NO: 1 - DNA, 1707 bp, Homo sapiens
  CDS (1)..(1707)
atgctcgccgcctccatcttccgtccgacactgctgctctgctggctggctgctccctggcccacccagcccgagagtctcttccacagccgggaccgctcggacctggagccgtccccactgcgccaggccaagcccattgccgacctccacgctgctcagcggttcctgtccagatacggctggtcaggggtgtgggcggcctgggggcccagtcccgaggggccgccggagacccccaagggcgccgccctggccgaggcggtgcgcaggttccagcgggcgaacgcgctgccggccagcggggagctggacgcggccaccctagcggccatgaaccggccgcgctgcggggtcccggacatgcgcccaccgcccccctccgccccgccttcgcccccgggcccgccccccagagcccgctccaggcgctccccgcgggcgccgctgtccttgtcccggcggggttggcagccccggggctaccccgacggcggagctgcccaggccttctccaagaggacgctgagctggcggctgctgggcgaggccctgagcagccaactgtccgtggccgaccagcggcgcattgtggcgctggccttcaggatgtggagcgaggtgacgccgctggacttccgcgaggacctggccgcccccggggccgcggtcgacatcaagctgggctttgggagaggccggcacctgggctgtccgcgggccttcgatgggagcgggcaggagtttgcacacgcctggcgcctaggtgacattcactttgacgacgacgagcacttcacacctcccaccagtgacacgggcatcagccttctcaaggtggccgtccatgaaattggccatgtcctgggcttgcctcacacctacaggacgggatccataatgcaaccaaattacattccccaggagcctgcctttgagttggactggtcagacaggaaagcaattcaaaagctgtatggctcctgtgagggatcatttgatactgcgtttgactggattcgcaaagagagaaaccaatatggagaggtgatggtgagatttagcacatatttcttccgtaacagctggtactggctttatgaaaatcgaaacaataggacacgctatggggaccctatccaaatcctcactggctggcctggaatcccaacacacaacatagatgcctttgttcacatctggacatggaaaagagatgaacgttatttttttcaaggaaatcaatactggagatatgacagtgacaaggatcaggccctcacagaagatgaacaaggaaaaagctatcccaaattgatttcagaaggatttcctggcatcccaagtcccctagacacggcgttttatgaccgaagacagaagttaatttacttcttcaaggagtcccttgtatttgcatttgatgtcaacagaaatcgagtacttaattcttatccaaagaggattactgaagtttttccagcagtaataccacaaaatcatcctttcagaaatatagattccgcttattactcctatgcatacaactccattttctttttcaaaggcaatgcatactggaaggtagttaatgacaaggacaaacaacagaattcctggcttcctgctaatggcttatttccaaaaaagtttatttcagagaagtggtttgatgtttgtgacgtccatatctccacactgaacatg

SEQ ID NO: 2 - protein, 569 aa, Homo sapiens
MLAASIFRPTLLLCWLAAPWPTQPESLFHSRDRSDLEPSPLRQAKPIADLHAAQRFLSRYGWSGVWAAWGPSPEGPPETPKGAALAEAVRRFQRANALPASGELDAATLAAMNRPRCGVPDMRPPPPSAPPSPPGPPPRARSRRSPRAPLSLSRRGWQPRGYPDGGAAQAFSKRTLSWRLLGEALSSQLSVADQRRIVALAFRMWSEVTPLDFREDLAAPGAAVDIKLGFGRGRHLGCPRAFDGSGQEFAHAWRLGDIHFDDDEHFTPPTSDTGISLLKVAVHEIGHVLGLPHTYRTGSIMQPNYIPQEPAFELDWSDRKAIQKLYGSCEGSFDTAFDWIRKERNQYGEVMVRFSTYFFRNSWYWLYENRNNRTRYGDPIQILTGWPGIPTHNIDAFVHIWTWKRDERYFFQGNQYWRYDSDKDQALTEDEQGKSYPKLISEGFPGIPSPLDTAFYDRRQKLIYFFKESLVFAFDVNRNRVLNSYPKRITEVFPAVIPQNHPFRNIDSAYYSYAYNSIFFFKGNAYWKVVNDKDKQQNSWLPANGLFPKKFISEKWFDVCDVHISTLNM

(19) United States
(12) Patent Application Publication
(10) Pub. No.: US 2002/0076800 A1
(43) Pub. Date: Jun. 20, 2002

(54) Protein

(76) Inventors: Natalie Samantha Delany, Stevenage (GB); Mark Robert Edbrooke, Stevenage (GB)

Correspondence Address: DAVID J LEVY, CORPORATE INTELLECTUAL PROPERTY, GLAXOSMITHKLINE, FIVE MOORE DR., PO BOX 13398, DURHAM, NC, 27709-3398, US

(21) Appl. No.: 09/931,147

(22) Filed: Aug. 16, 2001

(30) Foreign Application Priority Data
Aug. 17, 2000 (GB) 0020345.5

(51) Int. Cl. C12N 9/64; C07H 21/04; C12P 21/02; C12N 5/06

(52) U.S. Cl. 435/226; 435/69.1; 435/325; 435/320.1; 536/23.2

(57) ABSTRACT

The present invention provides an isolated matrix metalloprotease polypeptide comprising

(i) the amino acid sequence of SEQ ID NO: 2 or

(ii) a variant thereof which comprises a catalytic domain capable of binding a zinc residue or

(iii) a fragment of (i) or (ii) which comprises a catalytic domain capable of binding a zinc residue.

### CROSS REFERENCE TO RELATED APPLICATIONS

This application claims priority from Great Britain application number 0020345.5 filed on Aug. 17, 2000.

### FIELD OF THE INVENTION

The present invention relates to matrix metalloprotease polypeptides.

### BACKGROUND OF THE INVENTION

The matrix metalloproteinases (MMPs) are a family of zinc-dependent endoproteases responsible for controlled destruction of extracelluar matrix (ECM) components during normal physiological processes such as fetal development, wound healing, cellular migration and angiogenesis. Matrix degradation is a tightly regulated process, controlled by the relative levels of active MMPs and the naturally occurring tissue metalloproteinase inhibitors (TIMPs) which keep them in check. Disregulated MMP activity results in runaway ECM destruction, implicated in a wide variety of pathological conditions including arthritis, atherosclerosis, emphysema, ulceration, neurodegeneration and cancer metastasis. MMPs are multidomain proteins, typically containing an N-terminal prodomain, catalytic domain and C-terminal hemopexin or extracellular matrix binding domains. A subclass of the MMP family, the membrane type MMPs (MT-MMPs) possess a transmembrane domain near the C-terminus. The prodomain maintains the enzyme in an inactive state in the mature zymogen by binding the active site zinc residue via a conserved cysteine residue (the cysteine switch), normally positioned near the prodomain N-terminus. Active MMPs typically lack the prodomain, which is displaced from the active site and cleaved off during the activation process. The catalytic domain contains a conserved zinc binding sequence (HEXXH).

### SUMMARY OF THE INVENTION

A novel matrix metalloprotease, referred to herein as HIPHUM35, is now provided. HIPHUM35 is shown to be primarily expressed in adipose tissue, cerebellum, jejunum, lung, myometrium, omentum, prostate, small intestine and testis. Expression is upregulated in parasupranuclear palsy (PSP) brain, in chronic obstructive pulmonary disease (COPD) lung, VEGF treated endothelial cells and peripheral blood mononuclear cells (PBMCs). Expression is downregulated in colon tumour, breast tumour and lung carcinoma. The novel matrix metalloprotease is a screening target for the identification and development of novel pharmaceutical agents, including modulators of matrix metalloprotease activity. These agents may be used in the treatment and/or prophylaxis of CNS diseases such as parasupranuclear palsy (PSP), respiratory diseases such as chronic obstructive pulmonary disease (COPD), inflammatory respiratory diseases such as fibrotic diseases of the lung and cancers such as lung, colon, breast and endometrial carcinomas (DEC).

Accordingly, the present invention provides an isolated matrix metalloprotease polypeptide comprising

(i) the amino acid sequence of SEQ ID NO: 2;

(ii) a variant thereof which comprises a catalytic domain capable of binding a zinc residue; or

(iii) a fragment of (i) or (ii) which comprises a catalytic domain capable of binding a zinc residue.

According to another aspect of the invention there is provided a polynucleotide encoding a polypeptide of the invention which polynucleotide includes a sequence comprising:

(a) the nucleic acid sequence of SEQ ID NO: 1 and/or a sequence complementary thereto;

(b) a sequence which hybridises under stringent conditions to a sequence as defined in (a);

(c) a sequence that is degenerate as a result of the genetic code to a sequence as defined in (a) or (b); or

(d) a sequence having at least 65% identity to a sequence as defined in (a), (b) or (c).

The invention also provides:

an expression vector which comprises a polynucleotide of the invention and which is capable of expressing a polypeptide of the invention;

a host cell comprising an expression vector of the invention;

a method of producing a polypeptide of the invention which method comprises maintaining a host cell of the invention under conditions suitable for obtaining expression of the polypeptide and isolating the said polypeptide;

an antibody specific for a polypeptide of the invention;

a method for identification of a substance that modulates matrix metalloprotease activity and/or expression, which method comprises contacting a polypeptide, polynucleotide, expression vector or host cell of the invention with a test substance and determining the effect of the test substance on the activity and/or expression of the said polypeptide or the polypeptide encoded by the said polynucleotide, thereby to determine whether the test substance modulates matrix metalloprotease activity and/or expression;

a compound which modulates matrix metalloprotease activity and which is identifiable by the method referred to above;

a method of treating a subject having a disorder that is responsive to matrix metalloprotease stimulation or modulation, which method comprises administering to said subject an effective amount of substance of the invention; and

use of a substance that stimulates or modulates matrix metalloprotease activity in the manufacture of a medicament for the treatment or prophylaxis of a disorder that is responsive to stimulation or modulation of matrix metalloprotease activity.

Preferably the disorder is selected from CNS diseases such as parasupranuclear palsy (PSP), respiratory diseases such as chronic obstructive pulmonary disease (COPD), inflammatory respiratory diseases such as fibrotic diseases of the lung and cancers such as lung, colon, breast and endometrial carcinomas (DEC).

### BRIEF DESCRIPTION OF THE SEQUENCES

SEQ ID NO: 1 shows the nucleotide and amino acid sequences of human protein HIPHUM35.

SEQ ID NO: 2 is the amino acid sequence alone of HIPHUM35.

### BRIEF DESCRIPTION OF THE FIGURES

FIG. 1 shows αmacroglobulin capture by Hiphum 35, demonstrating that Hiphum 35 has protease activity.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to a human matrix metalloprotease, referred to herein as HIPHUM35, and variants thereof. Sequence information for HIPHUM35 is provided in SEQ ID NO: 1 (nucleotide and amino acid) and in SEQ ID NO: 2. A polypeptide of the invention thus consists essentially of the amino acid sequence of SEQ ID NO: 2 or of a variant of that sequence, or of a fragment of either thereof.

Polypeptides of the invention may be in a substantially isolated form. It will be understood that the polypeptide may be mixed with carriers or diluents which will not interfere with the intended purpose of the polypeptide and still be regarded as substantially isolated. A polypeptide of the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention. Routine methods, can be employed to purify and/or synthesise the proteins according to the invention. Such methods are well understood by persons skilled in the art, and include techniques such as those disclosed in Sambrook et al, Molecular Cloning: a Laboratory Manual, 2^{nd} Edition, CSH Laboratory Press, 1989, the disclosure of which is included herein in its entirety by way of reference.

The term “variant” refers to a polypeptide which has a same essential character or basic biological functionality as HIPHUM35. The essential character of HIPHUM35 can be defined as follows: HIPHUM35 is a matrix metalloprotease. Preferably a variant polypeptide is one which binds to the same extracellular matrix component as HIPHUM35. Preferably the polypeptide comprises a catalytic domain capable of binding a zinc residue. Typically the zinc residue binds to the consensus zinc binding sequence HEXXH, preferably HEIGH. Typically the catalytic domain is capable of cleaving a polypeptide when the metalloprotease is in an active state. The metalloprotease is in an active state when the polypeptide does not comprise a prodomain. Therefore, a polypeptide of the invention may comprise a prodomain and be in an inactive state or may lack a prodomain and be in an active state. A polypeptide comprising a prodomain may be cleaved to remove the prodomain and activate protease activity. Cleavage of the prodomain to activate the metalloprotease may occur as a result of the enzymatic activity of any suitable protease which may be a matrix metalloprotease or a protease of another family. The protease may comprise a mutated prodomain that does not function to maintain the polypeptide in an inactive state, for example because of mutation of the cysteine switch motif (PRCGVP) in the prodomain. The cysteine switch motif contains a cysteine residue which interacts with the zinc residue in the active site of the catalytic domain. Preferably a polypeptide of the invention comprises a hemopexin domain. Preferably a polypeptide of the invention binds to an extracellular matrix component. More preferably binding of a polypeptide of the invention to an extracellular matrix component occurs via a hemopexin domain.

Typically, a polypeptide of the invention is capable of cleaving a polypeptide such as a component of the extracellular matrix or a metalloprotease when activated. A polypeptide of the invention may be capable of binding a tissue inhibitor of metalloproteases (TIMP). Binding of a polypeptide of the invention to a TIMP typically inactivates metalloprotease activity. A polypeptide of the invention may be capable of activating a matrix metalloprotease by cleaving a prodomain.

A polypeptide having a same essential character as HIPHUM35 may be identified by monitoring for a function of the matrix metalloprotease selected from cleavage of extracellular matrix proteins such as collagen (including fibrillar collagen), elastin, laminin, vitronectin, fibronectin, enamel matrix, aggrecan, other matrix and basement membrane proteins and proteoglycans, cleavage of plasminogen and/or amyloid precursor protein (APP), cleavage of an artificial substrate such as a fluorescent substrate or colourimetric substrate and activation of MMPs or other proteases, either alone or in conjunction with other components (eg. TIMPs).

In another aspect of the invention, a variant is one which does not show the same activity as HIPHUM35 but is one which inhibits a basic function of HIPHUM35. For example, a variant polypeptide is one which inhibits protease activity of HIPHUM35, for example by binding to extracellular matrix component to prevent extracellular matrix component binding to HIPHUM35.

A typical assay for determining whether a polypeptide possesses HIPHUM35 protease activity comprises monitoring fluorescence or colourimetric changes of a fluorescent or colourimetric substrate of HIPHUM35 following incubation of the polypeptide with a said substrate.

Typically, polypeptides with more than about 70% identity preferably at least 80% or at least 90% and particularly preferably at least 95% at least 97% or at least 99% identity, with the amino acid sequences of SEQ ID NO: 2, are considered as variants of the proteins. Such variants may include allelic variants and the deletion, modification or addition of single amino acids or groups of amino acids within the protein sequence, as long as the peptide maintains a basic biological functionality of the HIPHUM35 receptor.

Amino acid substitutions may be made, for example from 1, 2 or 3 to 10, 20 or 30 substitutions. The modified polypeptide generally retains activity as a matrix metalloprotease. Conservative substitutions may be made, for example according to the following Table. Amino acids in the same block in the second column and preferably in the same line in the third column may be substituted for each other.

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

Shorter polypeptide sequences are within the scope of the invention. For example, a peptide of at least 20 amino acids or up to 50, 60, 70, 80, 100, 150, 200, 300 or 400 amino acids in length is considered to fall within the scope of the invention as long as it demonstrates a basic biological functionality of HIPHUM35. In particular, but not exclusively, this aspect of the invention encompasses the situation where the protein is a fragment of the complete protein sequence and may represent a catalytic domain or substrate binding domain. Preferred fragments include fragments comprising the cysteine switch domain found at amino acid residues 115 to 122 of full length HIPHUM35 and/or one or more of the haemopexin domain repeat elements found at amino acid residues 333 to 391, 394 to 449, 441 to 498 and 506 to 549 of full length HIPHUM35. Such fragments can be used to construct chimeric proteases preferably with another protease, more preferably with another member of the family of matrix metalloproteases. Such chimeric proteases may comprise different domains from different metalloproteases. For example, a fragment comprising the zinc protease domain of a polypeptide of the invention may be fused to a fragment of a different metalloprotease comprising one or more haemopexin repeat elements. The haemopexin domain of a chimeric protease may comprise one or more repeat elements from a polypeptide of the invention and one or more repeat elements from another metalloprotease.

Fragments of HIPHUM35 or a variant thereof can also be used to raise anti-HIPHUM35 antibodies. In this embodiment the fragment may comprise an epitope of the HIPHUM35 polypeptide and may otherwise not demonstrate the catalytic, substrate binding or other properties of HIPHUM35.

Polypeptides of the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Such modified polypeptides fall within the scope of the term “polypeptide” of the invention.

The invention also includes nucleotide sequences that encode for HIPHUM35 or a variant thereof as well as nucleotide sequences which are complementary thereto. The nucleotide sequence may be RNA or DNA including genomic DNA, synthetic DNA or cDNA. Preferably the nucleotide sequence is a DNA sequence and most preferably, a cDNA sequence. Nucleotide sequence information is provided in SEQ ID NO: 1. Such nucleotides can be isolated from human cells or synthesised according to methods well known in the art, as described by way of example in Sambrook et al, 1989.

Typically a polynucleotide of the invention comprises a contiguous sequence of nucleotides which is capable of hybridizing under selective conditions to the coding sequence or the complement of the coding sequence of SEQ ID NO: 1.

A polynucleotide of the invention can hydridize to the coding sequence or the complement of the coding sequence of SEQ ID NO: 1 at a level significantly above background. Background hybridization may occur, for example, because of other cDNAs present in a cDNA library. The signal level generated by the interaction between a polynucleotide of the invention and the coding sequence or complement of the coding sequence of SEQ ID NO: 1 is typically at least 10 fold, preferably at least 100 fold, as intense as interactions between other polynucleotides and the coding sequence of SEQ ID NO: 1. The intensity of interaction may be measured, for example, by radiolabelling the probe, e.g. with ³²P. Selective hybridisation may typically be achieved using conditions of medium to high stringency. However, such hybridisation may be carried out under any suitable conditions known in the art (see Sambrook et al, 1989. For example, if high stringency is required suitable conditions include from 0.1 to 0.2×SSC at 60° C. up to 65° C. If lower stringency is required suitable conditions include 2 ×SSC at 60° C.

The coding sequence of SEQ ID NO: 1 may be modified by nucleotide substitutions, for example from 1, 2 or 3 to 10, 25, 50 or 100 substitutions. The polynucleotide of SEQ ID NO: 1 may alternatively or additionally be modified by one or more insertions and/or deletions and/or by an extension at either or both ends. A polynucleotide may include one or more introns, for example may comprise genomic DNA. Additional sequences such as signal sequences which may assist in insertion of the polypeptide in a cell membrane may also be included. The modified polynucleotide generally encodes a polypeptide which has a HIPHUM35 activity. Alternatively, a polynucleotide encodes a catalytic or substrate-binding portion of a polypeptide or a polypeptide which inhibits HIPHUM35 activity. Degenerate substitutions may be made and/or substitutions may be made which would result in a conservative amino acid substitution when the modified sequence is translated, for example as shown in the Table above.

A nucleotide sequence which is capable of selectively hybridizing to the complement of the DNA coding sequence of SEQ ID NO: 1 will generally have at least 60%, at least 70%, at least 80%, at least 90%, at least 95%, at least 98% or at least 99% sequence identity to the coding sequence of SEQ ID NO: 1 over a region of at least 20, preferably at least 30, for instance at least 40, at least 60, more preferably at least 100 contiguous nucleotides or most preferably over the full length of SEQ ID NO: 1.

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul (1993) J. Mol. Evol. 36:290-300; Altschul et al (1990) J. Mol. Biol. 215:403-10.

Software for performing BLAST analyses is publicly available through the National Centre for Biotechnology Information (http://www.ncbi.nim.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, 1990). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The nucleotides according to the invention have utility in production of the proteins according to the invention, which may take place in vitro, in vivo or ex vivo. The nucleotides may be involved in recombinant protein synthesis or indeed as therapeutic agents in their own right, utilised in gene therapy techniques. Nucleotides complementary to those encoding HIPHUM35, or antisense sequences, may also be used in gene therapy.

Polynucleotides of the invention may be used as a primer, e.g. a PCR primer, a primer for an alternative amplification reaction, a probe e.g. labelled with a revealing label by conventional means using radioactive or non-radioactive labels, or the polynucleotides may be cloned into vectors.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700, 800, 900, 1000, 1250 or 1500 nucleotides in length, or even up to a few nucleotides, such as five or ten nucleotides, short of the coding sequence of SEQ ID NO: 1.

The present invention also includes expression vectors that comprise nucleotide sequences encoding the proteins or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. 1989.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used as test compounds in the assays of the invention or may be useful in a method of treatment of the human or animal body by therapy.

Preferably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with a origin of replication, optionally a promoter for the expression of the said polynucleotide and optionally a regulator of the promoter. The vectors may contain one or more selectable marker genes, for example an ampicillin resistence gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to polynucleotides which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

The invention also includes cells that have been modified to express the HIPHUM35 polypeptide or a variant thereof. Such cells include transient, or preferably stable higher eukaryotic cell lines, such as mammalian cells or insect cells, using for example a baculovirus expression system, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa, BHK, 3T3 and COS cells. Preferably the cell line selected will be one which is not only stable, but also allows for mature glycosylation of a polypeptide. Expression may be achieved in transformed oocytes. A polypeptide of the invention may be expressed in cells of a transgenic non-human animal, preferably a mouse. A transgenic non-human animal expressing a polypeptide of the invention is included within the scope of the invention. A polypeptide of the invention may also be expressed in Xenopus laevis oocytes.

A polypeptide for use in an assay of the invention may be overexpressed in bacterial cells, such as E. Coli, and isolated from the bacterial culture.

According to another aspect, the present invention also relates to antibodies, specific for a polypeptide of the invention. Such antibodies are for example useful in purification, isolation or screening methods involving immunoprecipitation techniques or, indeed, as therapeutic agents in their own right.

Antibodies may be raised against specific epitopes of the polypeptides according to the invention. Such antibodies may be used to block substrate binding to the receptor. An antibody, or other compound, “specifically binds” to a protein when it binds with preferential or high affinity to the protein for which it is specific but does substantially bind not bind or binds with only low affinity to other proteins. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

Antibodies of the invention may be antibodies to human polypeptides or fragments thereof. For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample, which method comprises:

I providing an antibody of the invention;

II incubating a biological sample with said antibody under conditions which allow for the formation of an antibody-antigen complex; and

III determining whether antibody-antigen complex comprising said antibody is formed.

A sample may be for example a tissue extract, blood, serum and saliva. Antibodies of the invention may be bound to a solid support and/or packaged into kits in a suitable container along with suitable reagents, controls, instructions, etc. Antibodies may be linked to a revealing label and thus may be suitable for use in methods of in vivo HIPHUM35 imaging.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

An important aspect of the present invention is the use of polypeptides according to the invention in screening methods. The screening methods may be used to identify substances that bind to matrix metalloprotease and in particular which bind to HIPHUM35 such as a substrate for the enzyme. Screening methods may also be used to identify agonists or antagonists which may modulate matrix metalloprotease activity, inhibitors or activators of HIPHUM35 activity, and/or agents which up-regulate or down-regulate HIPHUM35 expression.

Any suitable format may be used for the assay. In general terms such screening methods may involve contacting a polypeptide of the invention with a test substance and monitoring for binding of the test substance to the polypeptide or measuring protease activity. A polypeptide of the invention may be incubated with a test substance. Modulation of matrix metalloprotease activity may be determined. In a preferred aspect, the assay is a cell-based assay. Preferably the assay may be carried out in a single well of a microtitre plate. Assay formats which allow high throughput screening are preferred.

A typical assay for determining whether a test substance acts as an inhibitor or activator of HIPHUM35 activity comprises contacting a fluorescent or colourimetric substrate with a polypeptide of the invention and a test substance and monitoring protease activity by monitoring any change in the fluorescence or light emission. Any changes in the fluorescence of a substrate as a result of its proteolytic degradation by a polypeptide of the invention may be detected using a fluorescence plate reader. Colourimetic changes may be measured using a spectrophotometer. The inhibitory or stimulatory activity of a test substance may be determined by comparing any fluorescent or colourimetric changes observed in the presence of a test substance to any changes observed in the absence of a test substance and/or in the presence of a known inhibitor of HIPHUM35 activity.

Modulator activity can be determined by contacting cells expressing a polypeptide of the invention with a substance under investigation and by monitoring an effect mediated by the polypeptide. The cells expressing the polypeptide may be in vitro or in vivo. The polypeptide of the invention may be naturally or recombinantly expressed. Preferably, the assay is carried out in vitro using cells expressing recombinant polypeptide. Preferably, control experiments are carried out on cells which do not express the polypeptide of the invention to establish whether the observed responses are the result of activation of the polypeptide.

The binding of a test substance to a polypeptide of the invention can be determined directly. For example, a radiolabelled test substance can be incubated with the polypeptide of the invention and binding of the test substance to the polypeptide can be monitored. Typically, the radiolabelled test substance can be incubated with cell membranes containing the polypeptide until equilibrium is reached. The membranes can then be separated from a non-bound test substance and dissolved in scintillation fluid to allow the radioactive content to be determined by scintillation counting. Non-specific binding of the test substance may also be determined by carrying out a competitive binding assay.

Substances that inhibit the interaction of a polypeptide of the invention with a HIPHUM35 substrate or with another protease may also be identified through a yeast 2-hybrid assay or other protein interaction assay such as a co-immunoprecipitation or an ELISA based technique.

Assays may be carried out using cells expressing HIPHUM35, and incubating such cells with the test substance optionally in the presence of a HIPHUM35 substrate. The results of the assay are compared to the results obtained using the same assay in the absence of the test substance. Cells expressing HIPHUM35 constitutively may be provided for use in assays for HIPHUM35 function. Additional test substances may be introduced in any assay to look for inhibitors or activators of substrate binding or inhibitors or activators of protease activity.

Assays may also be carried out to identify substances which modify HIPHUM35 expression, for example substances which up- or down-regulate expression. Such assays may be carried out for example by using antibodies for HIPHUM35 to monitor levels of HIPHUM35 expression. Other assays which can be used to monitor the effect of a test substance on HIPHUM35 expression include using a reporter gene construct driven by the HIPHUM35 regulatory sequences as the promoter sequence and monitoring for expression of the reporter polypeptide.

Additional control experiments may be carried out.

Suitable test substances which can be tested in the above assays include combinatorial libraries, defined chemical entities and compounds, peptide and peptide mimetics, oligonucleotides and natural product libraries, such as display (e.g. phage display libraries) and antibody products.

Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Test substances may be used in an initial screen of, for example, 10 substances per reaction, and the substances of these batches which show inhibition or activation tested individually. Test substances may be used at a concentration of from 1 nM to 10 mM, preferably from 1 μm to 1 mM or from 1 μM to 100 μM, more preferably from 1 μM to 10 μM. Preferably, the activity of a test substance is compared to the activity shown by a known activator or inhibitor. A test substance which acts as an inhibitor may produce a 50% inhibition of activity of the receptor. Alternatively a test substance which acts as an activator may produce 50% of the maximal activity produced using a known activator.

Another aspect of the present invention is the use of polynucleotides encoding the HIPHUM35 polypeptides of the invention to identify mutations in HIPHUM35 genes which may be implicated in human disorders. Identification of such mutations may be used to assist in diagnosis or susceptibility to such disorders and in assessing the physiology of such disorders. Polynucleotides may also be used in hybridisation studies to monitor for up- or down-regulation of HIPHUM35 expression. Polynucleotides such as SEQ ID NO: 1 or fragments thereof may be used to identify allelic variants, genomic DNA and species variants.

The present invention provides a method for detecting variation in the expressed products encoded by HIPHUM35 genes. This may comprise determining the level of an HIPHUM35 expressed in cells or determining specific alterations in the expressed product. Sequences of interest for diagnostic purposes include, but are not limited to, the conserved portions as identified by sequence similarity and conservation of intron/exon structure. The diagnosis may be performed in conjunction with kindred studies to determine whether a mutation of interest co-segregates with disease phenotype in a family.

Diagnostic procedures may be performed on polynucleotides isolated from an individual or alternatively, may be performed in situ directly upon tissue sections (fixed and/or frozen) of patient tissue obtained from biopsies or resections, such that no nucleic acid purification is necessary. Appropriate procedures are described in, for example, Nuovo, G. J., 1992, “PCR In Situ Hybridization: Protocols And Applications”, Raven Press, N.Y.). Such analysis techniques include, DNA or RNA blotting analyses, single stranded conformational polymorphism analyses, in situ hybridization assays, and polymerase chain reaction analyses. Such analyses may reveal both quantitative aspects of the expression pattern of a HIPHUM35, and qualitative aspects of HIPHUM35 expression and/or composition.

Alternative diagnostic methods for the detection of HIPHUM35 nucleic acid molecules may involve their amplification, e.g. by PCR (the experimental embodiment set forth in U.S. Pat. No. 4,683,202), ligase chain reaction (Barany, 1991, Proc. Natl. Acad. Sci. USA 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. 15 USA 86:1173-1177), Q-Beta Replicase (Lizardi et al., 1988, Bio/Technology 6:1197) or any other nucleic acid amplification method, followed by the detection of the amplified molecules using techniques well known to those of skill in the art. These detection schemes are especially useful for the detection of nucleic acid molecules if such molecules are present in very low numbers.

Particularly suitable diagnostic methods are chip-based DNA technologies such as those described by Hacia et al., 1996, Nature Genetics 14:441-447 and Shoemaker et al., 1996, Nature Genetics 14:450-456. Briefly, these techniques involve quantitative methods for analyzing large numbers of nucleic acid sequence targets rapidly and accurately. By tagging with oligonucleotides or using fixed probe arrays, one can employ chip technology to segregate target molecules as high density arrays and screen these molecules on the basis of hybridization.

Following detection, the results seen in a given patient may be compared with a statistically significant reference group of normal patients and patients that have HIPHUM35 related pathologies. In this way, it is possible to correlate the amount or kind of HIPHUM35 encoded product detected with various clinical states or predisposition to clinical states.

Another aspect of the present invention is the use of the substances that have been identified by screening techniques referred to above in the treatment of disease states, which are responsive to regulation of matrix metalloprotease activity. The treatment may be therapeutic or prophylactic. The condition of a patient suffering from such a disease state can thus be improved.

In particular, such substances may be used in the treatment of CNS diseases such as parasupranuclear palsy (PSP), respiratory diseases such as chronic obstructive pulmonary disease (COPD), inflammatory respiratory diseases such as fibrotic diseases of the lung and cancers such as lung, colon, breast and endometrial carcinomas (DEC).

Substances that act as inhibitors of HIPHUM35 activity may be used in the treatment of disease states in which HIPHUM35 expression is up-regulated such as PSP or COPD. Substances that act as activators of HIPHUM35 activity may be used in the treatment of disease states in which expression of HIPHUM35 is down-regulated such as lung, colon or breast cancer.

Additional disease states that may be treated include atherosclerosis and multiple sclerosis (MS).

Substances identified according to the screening methods outlined above may be formulated with standard pharmaceutically acceptable carriers and/or excipients as is routine in the pharmaceutical art. For example, a suitable substance may be dissolved in physiological saline or water for injections. The exact nature of a formulation will depend upon several factors including the particular substance to be administered and the desired route of administration. Suitable types of formulation are fully described in Remington's Pharmaceutical Sciences, Mack Publishing Company, Eastern Pennsylvania, 17th Ed. 1985, the disclosure of which is included herein of its entirety by way of reference.

The substances may be administered by enteral or parenteral routes such as via oral, buccal, anal, pulmonary, intravenous, intra-arterial, intramuscular, intraperitoneal, topical or other appropriate administration routes.

A therapeutically effective amount of a modulator is administered to a patient. The dose of a modulator may be determined according to various parameters, especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Nucleic acid encoding HIPHUM35 or a variant thereof which inhibits HIPHUM35 activity may be administered to the mammal. In particular, a nucleic acid encoding a polypeptide with HIPHUM35 activity may be administered to a subject suffering from a condition in which HIPHUM35 expression is down-regulated, such as lung, colon or breast cancer. A nucleic acid encoding a variant of HIPHUM35 that inhibits HIPHUM35 activity may be administered to a patient suffering from a condition in which HIPHUM35 expression is up-regulated such as PSP or COPD. Nucleic acid, such as RNA or DNA, and preferably, DNA, is provided in the form of a vector, such as the polynucleotides described above, which may be expressed in the cells of the mammal.

Nucleic acid encoding the polypeptide may be administered by any available technique. For example, the nucleic acid may be introduced by needle injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to mucosal surfaces for example by intranasal, oral, intravaginal or intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

The following Examples illustrate the invention.

### EXAMPLE 1

### Characterisation of the Sequence

A matrix metalloprotease, designated as HIPHUM35 has been identified. The nucleotide and amino acid sequences of the receptor have been determined. These are set out below in SEQ ID NOs: 1 and 2. Suitable primers and probes were designed and used to analyse tissue expression. HIPHUM35 was found to be primarily expressed in adipose tissue, cerebellum, jejunum, lung, myometrium, omentum, prostate, small intestine and testis. Expression is upregulated in parasupranuclear palsy (PSP) brain, in chronic obstructive pulmonary disease (COPD) lung, VEGF treated endothelial cells and peripheral blood mononuclear cells (PBMCs). Expression is downregulated in colon tumour, breast tumour and lung carcinoma.

Original screens on normal and disease Taqman plates revealed significant expression profiles to link HIPHUM 35 with diseases such as CNS diseases, for example parasupranuclear palsy (PSP), respiratory diseases such as chronic obstructive pulmonary disease (COPD), inflammatory respiratory diseases such as fibrotic diseases of the lung and cancers such as lung, colon, breast.

HIPHUM 35 was found to be localised to chromosome 10q25-q26. This locus has been associated with the occurrence of cancers. One report details the presence of genes on 10q25-qter that are associated with proliferation that may be involved in the early steps of tumorigenesis of the breast (Ojopi EP. Rogatto SR. Caldeira JR. Barbieri-Neto J. Squire JA. Comparative genomic hybridization detects novel amplifications in fibroadenomas of the breast. Genes, Chromosomes & Cancer. 30(1):25-31, January 2001). It has also been suggest that tumour suppresser genes are localised on 10q25-q26, a locus that has been associated with patients suffering from endometrial carcinoma (Palmieri G. Manca A. Cossu A. Ruiu G. Pisano M. Cherchi P. Dessole S. Pintus A. Massarelli G. Tanda F. Pirastu M. Microsatellite analysis at 10q25-q26 in Sardinian patients with sporadic endometrial carcinoma: identification of specification patterns of genetic alteration. Cancer. 89(8):1773-82, Oct. 15, 2000). Furthermore, frequent loss of a specific chromosomic region in cancers is often associated with the inactivation of a tumor-suppressor gene. The long arm of chromosome 10 (10 q) is deleted in several types of tumour including squamous-cell carcinomas present in the respiratory tract (Gasparotto D. Vukosavljevic T. Piccinin S. Barzan L. Sulfaro S. Armellin M. Boiocchi M. Maestro R. Loss of heterozygosity at 10 q in tumors of the upper respiratory tract is associated with poor prognosis. International Journal of Cancer. 84(4):432-6, Aug. 20, 1999). A final report also details deletions on chromosome 10q25-q26 were highly significant for metastasising carcinoma's (Bockmuhl U. Petersen S. Schmidt S. Wolf G. Jahnke V. Dietel M. Petersen I. Patterns of chromosomal alterations in metastasizing and nonmetastasizing primary head and neck carcinomas. Cancer Research. 57(23):5213-6, Dec. 1, 1997).

As Taqman screening of the disease plate showed significant levels of expression in patients suffering from lung, breast and colon cancers, secondary plates were screened to analyse this further. The data collected from breast-, lung- and colon-tumour plates corroborated significant fold changes in HIPHUM 35 expression in tumour versus normal (or non tumour but taken from same patient) samples taken from cancer patients. The strongest correlation is measured from the breast tumour plate with expression levels for 11 out of 12 of the tumour samples being significantly different from normal (non-tumour) tissue (ie., significantly higher or lower). Similarly out of the 20 lung tumour samples, 10 contained significantly different levels of HIPHUM 35 from the normal samples and from the 19 colon tumour samples, significantly different levels of HIPHUM 35 were detected in 7 tumour samples in comparison to the equivalent normal samples.

As Taqman screening of the disease plate had showed significant levels of expression in lung, secondary plates were screened to analyse this further. The data collected from lung- and leukocyte-plates corroborated significant expression in cell types central to respiratory diseases as well as other inflammatory conditions. Hiphum35 is expressed in many haematopoietic cells including monocytes, macrophages, dendritic cells, B cells and differentiated T cells. As such Hiphum35 is implicated in the function of these cells, which are involved in many pathological responses in respiratory diseases such as asthma, COPD and allergic rhinitis, as well as a number of diseases including haemato-proliferative disorders such as lymphomas and leukemias; and conditions with an inflammatory component such as autoimmune diseases (for example rheumatoid arthritis, Crohn's, multiple sclerosis; lupus); and inflammatory bowel disease or osteoarthritis.

### EXAMPLE 2

### Screening for Substances Which Exhibit Protein Modulating Activity

Preparations of a purified polypeptide of the invention are generated for screening purposes. 96 and 384 well plate, high throughput screens (HTS) are employed using fluorescence or colourimetric indicator molecules, including but not limited to metalloprotease substrates such as GI 142612A (DNP-Pro-Cha-Gly-Cys(Me)-His-Als-Lys(NMA)-NH₂) and TES substrate (Ac-Pro-Leu-Gly-SCH[CH₂CH(CH₃)₂]CO-Leu-Gly-OC₂H₅) (Bachem). Also, αmacroglobulin trapping could be used as described in example 4 below. Secondary screening involves the same technology. Tertiary screens involve the study of modulators in rat, mouse and guinea-pig models of disease relevant to the target.

A brief screening assay protocol is as follows:—

A polypeptide of the invention is expressed in E. Coli, purified and refolded by direct dilution in assay buffer (200 mM NaCl, 50 mM Tris, 5 mM CaCl₂, 10 μM ZnSO₄, 0.01% Brij 35, pH 7.5). Test substances are provided in pools of 10 at 5 mM for each test substance for high throughput screening or are serially diluted in dose response assays. The screening assay is run on an automated system incorporating an OCRA rail to move the plates, a Tecan liquid handler, a Multimek liquid handler and a Titertek liquid handler. (The assay may also be run on manually or in combination with any suitable liquid handling equipment.)

60 μg of serially diluted test substance is added to 96 well plates (4 control wells with no inhibitor), 60 μl of a solution of a polypeptide of the invention (0.5 μM to 150 μM) is then added to each well and 50 mM EDTA (20 μl of 0.5M EDTA) is added to 4 inhibited control wells. The plates are then incubated at room temperature for 20 minutes before initiating with substrate (60 μl GI 142612A for a fluorescence assay or 10 μl of DNTB (5,5′-dithio-bis(2-nitrobenzoic acid) at 5 mM in 150 mM Tris base) followed by 10 μl TES substrate for a colourimetric assay). In a fluorescence assay, the plates are read in a Fluostar (SLT) fluorescence plate reader or equivalent at an excitation of 343 nm and an emission of 450 nm. In a colourimetric assay the plates are read continuously in a SLT spectrophotometer at 405 nm for 3 minutes.

Percentage inhibition is calculated for each concentration (unknown values=U) in the dose response based on the range determined using the value of the difference of the control (no test substance) (mean of 4=C1) and the EDTA treated wells (mean of 4=C2) using the equation 100*(1−(U−C2)/(C1−C2).

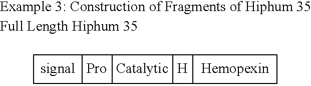

H=Hinge region

The signal domain runs from amino acid 0 to amino acid 39. The propetide domain runs from amino acid 40 to amino acid 142. The catalytic domain runs from amino acid 143 to amino acid 324. There is a polymorphism at position 191 of the catalytic domain. In Seq ID No: 1 this is shown as a valine, however in some individuals, this amino acid may be an alanine. The Hinge domain runs from amino acid 325 to amino acid 339. The Hemopexin domain runs from amino acid 340 to amino acid 569—the end of the polypeptide.

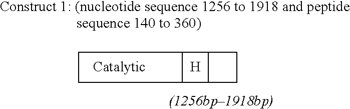

HipHum 35 construct no. 1 was designed to include primarily the catalytic domain. There is some extension beyond the catalytic domain into the hinge region and the construct terminates at the start of the hemopexin domain. Sole expression of the catalytic domain for other MMPs, such as MMP12, has resulted in active recombinant protein and the design of construct 1 is based on this work. Two versions have been prepared, one of which has a six histidine tag fused at the amino terminal to aid purification and the other is a native construct with no tag at all. Both have been designed to be expressed in a bacterial system.

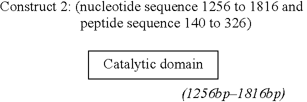

HipHum 35 construct no.2 is a modification of construct no.1. It was seen that construct 1 included a second cysteine residue located in the hinge region, the first cysteine being within the catalytic domain. There was a suggestion that this second cysteine residue could form di-sulphide bonds and prevent the correct re-folding of Hiphum 35 and cause the functionality of the recombinant protein to be affected. Construct 2 has been shortened by 102 nucleotides at the c terminal end in order to remove the cysteine and now terminates two residues into the hinge region. A six histidine tag has been fused to the amino terminal and as before the construct has been designed to be expressed in a bacterial system.

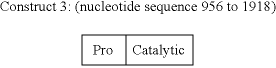

The closest human family member to Hiphum 35 is MMP 19 and this protease has been successfully expressed and characterised by constructing a recombinant protein comprising the propeptide and catalytic domain. In order to enhance correct protein folding and activity construct 3 is based on MMP19 work and is being designed to include both the pro and catalytic domains.

### EXAMPLE 4

### Evidence for Protease Activity

Protease activity can be confirmed by α₂Macroglobulin trapping, see Loechel, F et al. (1999) J. Biol. Chem. 274, 13427-13433. Human α2Macroglobulin is a plasma glycoprotein with a molecular mass of 725 kDa and consists of four identical subunits of about 180 kDa. It binds to and inhibits most endopeptidases regardless of their substrate specificity.

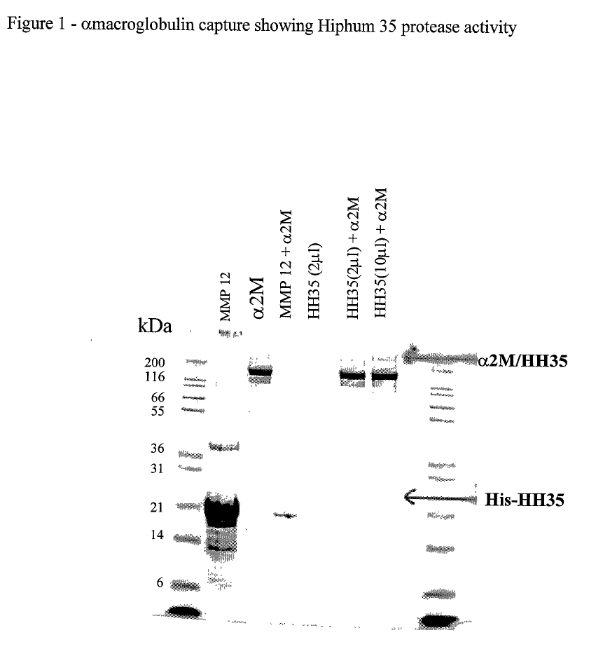

The protease cleaves a ‘bait’ region of α₂M located near the middle of the subunit. This event triggers a large conformational change in α₂M and entraps the protease. The protein gel shown in FIG. 1 shows an additional band of>200 kDa in the HH35/α₂M sample, this could represent HH35 entrapped within an α₂M subunit. The recombinant HH35 protein used in this assay included the catalytic domain only, ie construct 2.

What is claimed is:

1. An isolated matrix metalloprotease polypeptide comprising
(i) the amino acid sequence of SEQ ID NO: 2 or
(ii) a variant thereof which comprises a catalytic domain capable of binding a zinc residue or
(iii) a fragment of (i) or (ii) which comprises a catalytic domain capable of binding a zinc residue.

2. A polypeptide according to claim 1 wherein the variant (ii) has at least 80% identity to the amino acid sequence of SEQ ID NO: 2.

3. A polynucleotide encoding a polypeptide according to claim 1.

4. A polynucleotide according to claim 3 which is a cDNA sequence.

5. A polynucleotide encoding a matrix metalloprotease polypeptide which comprises a catalytic domain capable of binding a zinc residue which polynucleotide comprises:
(a) the nucleic acid sequence of SEQ ID NO: 1 and/or a sequence complementary thereto;
(b) a sequence which hybridises under stringent conditions to a sequence as defined in (a);
(c) a sequence that is degenerate as a result of the genetic code to a sequence as defined in (a) or (b); or
(d) a sequence having at least 65% identity to a sequence as defined in (a), (b) or (c).

6. An expression vector comprising a polynucleotide according to claim 3.

7. A host cell comprising an expression vector according to claim 6.

8. An antibody specific for a polypeptide according to claim 1.

9. A method for the identification of a substance that modulates matrix metalloprotease activity and/or expression, which method comprises:
(i) contacting a test substance and a polypeptide according to claim 1 or 2, a polynucleotide according to any one of claims 3 to 5, an expression vector according to claim 6 or a host cell according to claim 7, and
(ii) determining the effect of the test substance on the activity and/or expression of the said polypeptide or the polypeptide encoded by said polynucleotide, thereby to determine whether the test substance modulates matrix metalloprotease activity and/or expression.

10. A method according to claim 9 wherein the polypeptide is in a substantially isolated form.

11. A substance which modulates matrix metalloprotease activity and which is identifiable by a method according to claim 9.

12. A method of treating a subject having a disorder that is responsive to matrix metalloprotease modulation, which method comprises administering to said subject an effective amount of a substance according to claim 11.

13. A method according to claim 12 wherein the disorder is selected from CNS diseases such as parasupranuclear palsy (PSP), respiratory diseases such as chronic obstructive pulmonary disease (COPD), inflammatory respiratory diseases such as fibrotic diseases of the lung and cancers such as lung, colon, breast and endometrial carcinomas (DEC).

14. Use of a substance as defined in claim 11 in the manufacture of a medicament for treatment or prophylaxis of a disorder that is responsive to stimulation or modulation of matrix metalloprotease activity.

15. A use according to claim 14 wherein the disorder is selected from CNS diseases such as parasupranuclear palsy (PSP), respiratory diseases such as chronic obstructive pulmonary disease (COPD), inflammatory respiratory diseases such as fibrotic diseases of the lung and cancers such as lung, colon, breast and endometrial carcinomas (DEC).

16. A method of producing a polypeptide having the amino acid sequence of SEQ ID NO:2, which method comprises maintaining a host cell as claimed in claim 7, said method conducted under conditions suitable for obtaining expression of said polypeptide and isolating said polypeptide.
